(19) United States
(12) Patent Application Publication
(10) Pub. No.: US 2009/0181010 A1
(43) Pub. Date: Jul. 16, 2009

(54) Arginase II: a target for the prevention of atherosclerosis

(75) Inventors: Dan E. Berkowitz, Baltimore, MD (US); Sungwoo Ryoo, Timonium, MD (US); Artin A. Shoukas, Baltimore, MD (US); Gaurav Gupta, McLean, VA (US); Lewis Romer, Baltimore, MD (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 12/284,723

(22) Filed: Sep. 23, 2008

Related U.S. Application Data
(63) Continuation of application No. PCT/US2006/026116, filed on Jun. 29, 2006.
(60) Provisional application No. 60/785,315, filed on Mar. 23, 2006.

(51) Int. Cl. A61K 39/395; A61K 31/7088; A61K 38/00; C12Q 1/34

(52) U.S. Cl. 424/130.1; 514/44; 514/2; 435/18

(57) ABSTRACT

The instant invention provides methods and compositions for the treatment of. atherosclerotic disease. Specifically, the invention provides methods and compositions for modulating the activity of Arginase II, the production of Arginase II or the amount of free Arginase II for the treatment of atherosclerotic disease.

### RELATED APPLICATIONS

The instant invention claims the benefit of U.S. Provisional Application No. 60/785,315, filed Mar. 23, 2006, the entire contents of which are expressly incorporated herein by reference.

### GOVERNMENT SUPPORT

The following invention was supported at least in part by NIH Grants AG021523, HL058064 and AI061042. Accordingly, the government may have certain rights in the invention.

### BACKGROUND OF THE INVENTION

Atherosclerosis is a disorder characterized by cellular changes in the arterial intima and the formation of arterial plaques containing intracellular and extracellular deposits of lipids. The thickening of artery walls and the narrowing of the arterial lumen underlies the pathologic condition in most cases of coronary artery disease, aortic aneurysm, peripheral vascular disease, and stroke. A number of metabolic pathways and a cascade of molecular events is involved in the cellular morphogenesis, proliferation, and cellular migration that results in atherogenesis (Libby et al. (1997) Int J Cardiol 62 (S2):23-29).

The artery walls consist of three layers: the intima (innermost), the media, and the adventitia (outermost). The intima consists of a layer of endothelial cells lining the lumen of arteries and arterioles. Endothelial cells form a barrier against the indiscriminate entry of substances from the blood into the artery. Specific transporter proteins expressed by endothelial cells facilitate barrier function. Endothelial cells also secrete a number of substances which help regulate downstream vascular contractility blood coagulation, and other aspects of vascular biology. The medial layer of the arterial wall contains smooth muscle cells in a matrix of collagen and elastic fibers produced by the smooth muscle cells. Contraction and relaxation of the smooth muscle layer allows arteries and arterioles to modulate blood pressure and blood flow. The outermost layer of the arterial wall, the adventitia, is a mixture of collagen bundles, elastic fibers, some smooth muscle cells, fibroblasts and nerve cells. The adventitia provides structural integrity to the blood vessel and acts as a support matrix for the media and intima.

Initiation of an atherosclerotic lesion often occurs following vascular endothelial cell injury often attributable to hypertension, diabetes mellitus, hyperlipidemia, fluctuating shear stress, smoking, or transplant rejection.

The initiation and progression of atherosclerotic lesion development requires the interplay of various molecular pathways. Many genes that participate in these processes are known, and some of them have been shown to have a direct role in atherosclerosis pathogenesis by animal model experiments, in vitro assays, and epidemiological studies (Krettek et al. (1997) Arterioscler Thromb Vasc Biol 17:2897-2903; Fisher et al. (1997) Atherosclerosis 135:145-159; Shih et al. (1998) Circulation 95:2684-2693; and Bocan et al. (1998) Atherosclerosis 139:21-30).

The idea that endothelium derived nitric oxide (NO) is an important molecule in the prevention of the development as well as in the prevention of progression of atherosclerosis is well established (Arterioscler Thromb Vasc Biol. 2006; 26:267-271. and Circulation. 2006; 113:1708-1714). NO is a potent vasodilator, inhibitor of platelet and leucocyte adhesion, inhibitor of vascular smooth muscle proliferation. Decreased NO bioavailability leads to a loss of these NO mediated effects all of which contribute to the atherodegenerative process.

A need exists for new and improved targets for the treatment of atherosclerotic disease. Moreover, new and improved therapeutics for the treatment of atherosclerotic disease and prognostic methods are needed to combat the increasing rate of atherosclerotic disease. Since the endothelium is the critical “organ” through which risk factors such as increased cholesterol and smoking are mediated therapuetoic strategies aimed at pathways that promote protective endothelial dependent NO production (independent of risk modification) represent promising modalities for the treatment of this highly prevalent disease.

### SUMMARY OF THE INVENTION

The instant invention is based, at least in part, on the discovery that oxLDL causes an upregulation of Arginase II. Arginase II upregulation leads to a decrease in vasoprotective NO production (and increase in reactive oxygen species production) due to L-arginine depletion and endothelial nitric oxide synthase “uncoupling”. Therefore, Arginase II is an important regulator of events leading up to atherosclerotic disease.

Accordingly, in one aspect, the instant invention provides methods of treating or preventing atherosclerotic disease in a subject by administering to the subject an effective amount of a compound inhibits the expression of Arginase II, the activity of Arginase II, or level of free of Arginase II, thereby treating or preventing atherosclerotic disease in a subject.

In a specific embodiment, the compound inhibits the level of free Arginase II. In an related embodiment, the compound inhibits the level of free Arginase II by inhibiting the dissociation of Arginase II from microtubules. In a specific embodiment, the compound is a microtubule stabilizing agent, e.g. paclitaxel, Doublecortin, epothilone, Laulimalide, Vincristine or Epothilone B. In another embodiment, the compound is an antibody.

In a related embodiment, the level of free Arginase II is inhibited by decreasing the amount of oxLDL in a cell, e.g., plasma oxLDL.

In another embodiment, the compound decreases the transcription or translation of Arginase II. In a specific embodiment, the compound decreases the translation of Arginase II. In specific embodiments, the compound that decreases the translation of Arginase II is a nucleic acid molecule, e.g., an antisense RNA molecule, a siRNA molecule or a shRNA molecule. In a specific embodiment, the molecule is an siRNA molecule comprising the sequence set forth as SEQ ID NO:3.

In another embodiment, the compound inhibits the activity of Arginase II. In a related embodiment, the compound is a small molecule, peptide, polypeptide, or nucleic acid molecule.

In another embodiment, the atherosclerotic disease is oxLDL dependent atherosclerotic disease.

In another aspect, the instant invention provides methods of treating a subject having endothelial dysfunction by administering to the subject an effective amount of a compound inhibits the expression of Arginase II, the activity of Arginase II, or level of free of Arginase II, thereby treating a subject having endothelial dysfunction.

In one embodiment, the compound inhibits the level of free Arginase II. In a related embodiment, the compound inhibits the level of free Arginase II by inhibiting the dissociation of Arginase II from microtubules. In a further related embodiment, the compound is a microtubule stabilizing agent, e.g., palitaxel, Doublecortin, epothilone, Laulimalide, Vincristine or Epothilone B. In another embodiment, the compound is an antibody.

In another embodiment, the level of free Arginase II is inhibited by decreasing the amount of oxLDL in a cell, e.g., plasma oxLDL.

In another embodiment, the compound decreases the transcription or translation of Arginase II. In a specific embodiment, the compound decreases the translation of Arginase II. In one embodiment, the compound that decreases the translation of Arginase II is a nucleic acid molecule, e.g., an antisense RNA molecule, a siRNA molecule or a shRNA molecule. In a specific embodiment, the nucleic acid molecule is an siRNA molecule comprising the sequence set forth as SEQ ID NO:3.

In another embodiment, the compound inhibits the activity of Arginase II, e.g., a small molecule, peptide, polypeptide, or nucleic acid molecule.

In another embodiment, the atherosclerotic disease is oxLDL dependent atherosclerotic disease.

In another embodiment, the inhibition of Arginase II results in a increase in nitric oxide (NO) production.

In another aspect, the instant invention provides methods of determining if a subject is at risk of developing atherosclerotic disease by obtaining a biological sample from the subject and determining the level of free Arginase II in the sample, wherein an elevated level of free Arginase II in the sample as compared to a control level is indicative that the subject is at risk of developing atherosclerotic disease.

In one embodiment, the level of free Arginase II is determined by cellular imaging using a detectable antibody. In another embodiment, the antibody is specific for free Arginase II. The antibody can be, for example, a monoclonal, polyclonal, humanized, human, or chimeric antibody, or a fragment thereof.

In another embodiment, the method further comprises the use of a detectable antibody that is specific for tubulin.

In another embodiment, the biological sample comprises cardiac myocytes.

In another aspect, the instant invention provides methods for treating or preventing atherosclerotic disease by modulating the activity of Arginase II comprising contacting the Arginase II polypeptide or a cell expressing the Arginase II polypeptide with a compound which binds to Arginase II in a sufficient concentration to modulate the activity of the to Arginase II.

In another aspect, the instant invention provides methods for identifying a compound which modulates the activity or location of Arginase II by contacting Arginase II, or a cell expressing Arginase II with a test compound; and determining whether the test compound binds to Arginase II. In a related embodiment, the modulation of Arginase II is detected by detection of a change in the rate of Arginase II enzyme activity of detection of an increase or decrease in free Arginase II in a cell.

In another related embodiment the method is for the treatment or prevention of atherosclerotic disease.

In another aspect, the instant invention provides methods for identifying a compound which treats or prevents atherosclerotic disease by modulating the activity of Arginase II comprising contacting Arginase II with a test compound, and determining the effect of the test compound on the activity of the Arginase II to thereby identify a compound which modulates the activity Arginase II and treats or prevents atherosclerotic disease.

In another aspect, the invention provides compounds for the treatment of atherosclerotic disease, wherein the compounds are identified by the methods described herein.

The invention further provides pharmaceutical compositions comprising the compound identified by the methods disclosed herein.

In another aspect, the invention provides kits comprising a compound or pharmaceutical composition of the invention and instructions for use. In another embodiment, the kit is for the treatment of atherosclerotic disease.

In another aspect, the invention provides kits for the diagnosis of atherosclerotic disease comprising an antibody specific for Arginase II, and instructions for use. In one embodiment, the antibody further comprises a detectable label. In another embodiment, the kit further comprises an antibody specific for tubulin. In a further embodiment, the antibody further comprises a detectable label.

### DESCRIPTION OF THE DRAWINGS

FIGS. 1A-B depict OxLDL increase arginase activity in a time- and a dose-dependent manner. A) HAECs were incubated with 50 μg/ml of OxLDL for 5 m to 24 h. Cellular arginase activity was then measured as described in methods section (n=9 from 3 different experiments, ANOVA analysis, p<0.0001). B) HAECs were incubated with increasing doses (5-100 μg/ml) of OxLDL for 10 min after which cellular arginase activity was measured (n=9 from 3 separate experiments, ANOVA analysis, p<0.0001).

FIG. 2 depicts an increase in arginase activity is associated with a reciprocal decrease in endothelial cell NO production. HAECs were stimulated with 50 μg/ml of OxLDL in presence of or absence of arginase inhibitor, BEC, after which the cellular NOx was measured. OxLDL resulted in a significant time-dependent decrease in endothelial cell NO production (p=0.0018 vs. untreated cells). Preincubation of endothelial cells with BEC (10 μmol/L) prevented the OxLDL-induced decrease in NO production. (n=6 from 3 experiments). The decreased NO production by OxLDL stimulation was associated with a proportionate decrease in total eNOS protein levels.

FIGS. 3A-D demonstrate that Arginase II is the primary arginase isoform expressed in HAECs. A) RT-PCR was performed with isoform-specific primers arginase I and arginase II on mRNA isolated from cells at baseline and following OxLDL stimulation at different time intervals. Arginase II, but not arginase I, was expressed in HAECs both at baseline and following OxLDL stimulation (n=3). B) SiRNA targeted to arginase II was transfected into HAEC by Oligofectamine reagent. Incubation of SiRNA (6.6 and 25 μmol) for 36 hours significantly decreased arginase II protein levels. Furthermore SiRNA pre-incubation prevented the OxLDL-induced increase in arginase II (*p=0.046, **p=0.0017 vs. untreated cells, #p=0.0117, ##p=0.0025 vs. OxLDL; n=3). C) Decreased arginase II levels after SiRNA transfection were associated with a proportionate decrease in arginase enzyme activity (*p<0.0001, **p<0.0001 vs. untreated cells; #p<0.0001 vs OxLDL, n=6), indicating that arginase II is the predominant enzymatically active isoform in HAEC. D) NOS activity was significantly increased after treatment with either 6.6 or 25 pmol/L of arginase II-specific SiRNA. (*p<0.0001, **p=0.0055, ***p<0.0001 vs. untreated cells; #p=0.0012, #Hp=0.0002 vs. OxLDL; n=6).

FIGS. 4A-B demonstrate transcriptional Induction and translational activation of arginase II by OxLDL. A) Quantitative PCR with arginase II specific primers was performed at different time intervals following OxLDL stimulation. There was a significant increase in arginase II expression at 4 hrs (2.58-fold, *p<0.0001 vs. untreated cells). This was completely inhibited by actinomysin D (10 μg/ml; 1.0±0.06 (control) vs. 1.1±0.15 (Act D)). #p=0.0002 vs. OxLDL 4 hrs, n=6 from 2 different experiments. (B) Protein level of arginase II was analyzed following OxLDL stimulation for different times. The arginase II protein was significantly increased at 12 hrs following OxLDL stimulation (*p=0.0015 vs. untreated cells), which was completely blocked by cycloheximide (10 μM) incubation (n=3 different experiments). # vs. OxLDL 12 hrs, p=0.0011.

FIGS. 5A-I depict colocalization of arginase II with microtubules in HAECs. Immunofluorescence images of beta-tubulin (green, A, D, G) and arginase II (red, B, E, H) in untreated cells (A-C), OxLDL-treated cells (50 μg/ml, 2 hours) (D-F), and Nocodazole-treated cells (50 μM, 30 minutes) (G-I). Merged images are shown in C, F, and I. In untreated cells, arginase II and tubulin are colocalized (C; colocalization appears yellow). Treatment with OxLDL leads to diffuse arginase localization (E) and disruption of the tubulin-Arg association (F). Treatment with nocodazole causes microtubule depolymerization (G) as well as diffuse arginase localization (H).

FIGS. 6A-D depict OxLDL increase microtubule depolymerization and arginase activity. Tubulin depolymerization assays were used to separate cell lysates into fractions of soluble (cytosolic) tubulin and insoluble (polymerized) tubulin. (A) Treatment of lysates with OxLDL (50 μg/ml, 30 minutes) and/or nocodazole resulted in redistribution of tubulin and arginase II from the insoluble to the soluble fraction. This redistribution was prevented by the microtubule-stabilizing agent, epothilone B (0.1 μM, 30 minutes). n=4 different experiments. *, #p<0.0001 vs. untreated cells; **, (0.1 μM, 30 minutes). n=4 different experiments. *, #p<0.0001 vs. untreated cells; **, ##p<0.0001 vs. Nocodazole treated cells. (B) Arginase II was co-immunoprecipitated with microtubular protein. Tubulin-stabilized cell lysates were immunoprecipitated with anti-tubulin antibody and immunoblotted with arginase II antibody. (C) Treatment of HAEC with either OxLDL or Nocodazole led to an increase in arginase activity and a corresponding decrease in NOS activity. (*p<0.0001 vs. untreated cells; # p<0.0001; n=6). (D) OxLDL effects on arginase activity and NOS activity are blocked by the microtubule-stabilizing agent epothilone B (*p=0.0002, **p<0.0001 vs. untreated cells; #p=0.0015 vs, OxLDL, n=6).

FIGS. 7A-B depict arginase dependent endothelial dysfunction in OxLDL treated rat aorta. Arginase inhibition restores endothelial function and increases NO production in rat aortic rings. A) Incubation of rat aortic rings with Ox-LDL (overnight˜16 hrs) resulted in a significant increase in arginase activity in endothelium intact (E+) rings (*p<0.0001 vs. E+ untreated control; n=5) but not in rings in which the endothelium had been denuded (E−). The increase in arginase activity was blocked by preincubation with BEC (10 μml/L, *p=0.0007, **p<0.0001 vs. untreated control; #p=0.0003 vs. OxLDL; n=5). B) Increased arginase activity was associated with a reciprocal decrease in NO production in E+ rings (*p<0.0001 vs. E+ untreated control; n=5) and a decreased NO production was inhibited following preincubation with BEC (*p<0.0001, **p=0.0019 vs. untreated control; #p<0.0001 vs. OxLDL; n=5).

FIGS. 8A-B Arginase inhibition decreases vascular stiffness and restores endothelial function in Apo E knockout mice. 16 WT mice were randomized to receive a normal or high cholesterol (HC) diet. 16 KO mice all fed a HC diet were randomized to receive either 4 weeks of S-(2-boronethyl)-L-cysteine (BEC), a selective, slow binding, reversible competitive transition state inhibitor of arginase [Ki=210 nM (human arginase II)], (1 mg/mice) or placebo (water) (W) by osmotic infusion pump (Alzet). Aortic Pulse wave velocity (PWV) (measured by 20 MHz pulsed Doppler from arch to abdominal aorta (4 cm) was used to measure vascular stiffness before and after pump implanatation.

A) Aortic arginase activity was significantly increased in KO mice compared to WT. This was associated with a significant decrease in NO production (measured by griess method). BEC treated mice had a significant decrease in arginase activity with an associated restoration of NO production to WT. The increase in arginase activity in Apo E mice was associated with an increase in arginase II abundance (B). PWV was significantly increased in KO compared to WT (4.8±0.25 vs 3.9±0.06 m/sec, p <0.002). Furthermore WT mice fed HC increased vascular stiffness (PWV 3.8±0.19 vs 4.5±0.52 m/sec, p <0.03) compared with normal diet (3.8±0.11 vs 4.0±0.11 m/sec, p=NS). Placebo treated KO mice had no changes in PWV (4.7±0.29 Versus 4.8±0.29 m/sec, p=NS) In marked contrast, KO mice treated with BEC demonstrated a dramatic decrease in PWV (4.9±0.22 vs 4.0±0.25 m/sec, p <0.03) such that they were not significantly different from WT/normal diet. Thus the effects of OxLDL on arginase activity occurs both in isolated endothelial cells, vascular tissue in vitro as well as in animals in vivo. Furthermore pharmacologic inhibition of arginase II in vivo can restore vascular compliance to normal in atherogenic prone mice with known endothelial dysfunction.

### DETAILED DESCRIPTION OF THE INVENTION

The endothelium plays a central role in overall vascular homeostasis including modulating vasoactivity, platelet activation, leukocyte adhesion and smooth muscle cell proliferation and migration. Endothelial nitric oxide (NO) is a major mediator of these effects, and impaired NO signaling is considered an early marker of the atherodegenerative process.

Endothelial cells (EC) have the capacity to internalize LDLs via cell surface LDL receptors and then oxidize LDLs to form OxLDL which can induce adhesion molecule expression^{1,2}, superoxide anion formation³, EC apoptosis^{4,5}, and impair endothelial NO formation^{6,7}.

Nitric oxide (NO) is produced by the action of endothelial nitric oxide synthase (eNOS) which utilizes L-arginine as its substrate. Arginase is present in two isoforms, arginase I or the hepatic isoform and arginase II or the extra-hepatic (mitochondrial) isoform, each of which are encoded by distinct genes^{10,11}. Arginase I catalyzes the final step of the urea cycle in hepatocytes. However, recent studies in other tissues demonstrate that arginase I expression can be induced by LPS, IL-13, and hypoxia¹²⁻¹⁶. L-ornithine, the product of arginase II is essential in the synthesis of polyamines, peptides that modulate cell proliferation and differentiation¹⁷. Importantly, both arginase isoforms have been shown to reciprocally regulate NO production. Arginase I regulates NO production in rat aortic endothelial cells¹⁸ and macrophages¹⁹. Arginase II reciprocally regulates penile NO production modulating erectile function²⁰ and is upregulated by thrombin stimulation in human umbilical vein endothelial cells (HUVEC) via a Rho pathway-dependent mechanism²¹.

The instant invention is based, at least in part, on the discovery that oxLDL causes an upregulation of Arginase II. Arginase II reciprocally regulates NO production by endothelial cells. Accordingly, Arginase II is an important regulator of events leading up to atherosclerotic disease.

As used herein the term “atherosclerotic disease” is intended to include diseases and disorders of the arteries. These diseases and disorders are often characterized by hardening of the arteries. Disorders associated with atherosclerotic disease can include, for example, myocardial infarction, stroke, angina pectoris and peripheral arteriovascular disease.

As used herein, the term “endothelial dysfunction” is intended to mean the earliest measurable functional abnormality of the vessel wall. Endothelial Dysfunction is closely related to the risk factors of atherosclerosis, to their intensity and duration. Endothelial dysfunction is also occurs in subjects having type I and II diabetes systemic lupus erythematosus, septic shock, hypertension, hypercholesterolaemia, diabetes as well as from environmental factors, such as from smoking tobacco products. For the most part impired NO signaling in the major contributor to endothelial dysfunction (Circulation. 2006; 113:1708-1714).

Accordingly, in one aspect, the invention provides methods (also referred to herein as “screening assays”) for identifying modulators, i.e., candidate or test compounds or agents (e.g., peptides, peptidomimetics, small molecules or other drugs) which bind to Arginase II proteins or have a inhibitory effect on, for example, the expression, activity or the amount of free Arginase II. In alternative embodiments, the test compounds are compounds can be compounds that stabilize microtubules thereby inhibiting the release of Arginase II from the microtubules. The compounds tested as modulators of Arginase II can be any small organic molecule, or a biological entity, such as a protein, e.g., an antibody or peptide, a sugar, a nucleic acid, e.g., an antisense oligonucleotide, RNAi, or a ribozyme, or a lipid. Typically, test compounds will be small organic molecules, peptides, lipids, and lipid analogs. Exemplary Arginase II inhibitors that are known in the art include, e.g., N-hydroxay-nor-L-arginine (Nor-NOHA) and S-(2-boronoethyl)-L-cysteine (BEC).

In one embodiment, the invention provides assays for screening candidate or test compounds which are substrates of an Arginase II protein or polypeptide or biologically active portion thereof. In another embodiment, the invention provides assays for screening candidate or test compounds which bind to or modulate the activity of an Arginase II protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses an Arginase II protein or biologically active portion thereof is contacted with a test compound and the ability of the test compound to modulate Arginase II activity is determined. Determining the ability of the test compound to modulate Arginase II activity can be accomplished by monitoring, for example, intracellular calcium, IP3, or diacylglycerol concentration, phosphorylation profile of intracellular proteins, cell proliferation and/or migration, or the activity of an Arginase II-regulated transcription factor. The cell, for example, can be of mammalian origin, e.g., an endothelial cell. Alternatively, the ability of the test compound to inhibit release of Arginase II from the microtubules can be evaluated.

The ability of the test compound to modulate Arginase II binding to a substrate or to bind to Arginase II can also be determined. Determining the ability of the test compound to modulate Arginase II binding to a substrate can be accomplished, for example, by coupling the Arginase II substrate with a radioisotope or enzymatic label such that binding of the Arginase II substrate to Arginase II can be determined by detecting the labeled Arginase II substrate in a complex. Alternatively, Arginase II could be coupled with a radioisotope or enzymatic label to monitor the ability of a test compound to modulate Arginase II binding to a Arginase II substrate in a complex. Determining the ability of the test compound to bind Arginase II can be accomplished, for example, by coupling the compound with a radioisotope or enzymatic label such that binding of the compound to Arginase II can be determined by detecting the labeled Arginase II compound in a complex. For example, compounds (e.g., Arginase II substrates) can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., an Arginase II substrate) to interact with Arginase II without the labeling of any of the interactants. For example, a microphysiometer can be used to detect the interaction of a compound with Arginase II without the labeling of either the compound or the Arginase II. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and Arginase II.

In another embodiment, an assay is a cell-based assay comprising contacting a cell expressing an Arginase II target molecule (e.g., an Arginase II substrate) with a test compound and determining the ability of the test compound to modulate (e.g., stimulate or inhibit) the activity of the Arginase II target molecule. Determining the ability of the test compound to modulate the activity of an Arginase II target molecule can be accomplished, for example, by determining the ability of the Arginase II protein to bind to or interact with the Arginase II target molecule.

Determining the ability of the Arginase II protein or a biologically active fragment thereof, to bind to or interact with an Arginase II target molecule can be accomplished by one of the methods described above for determining direct binding. In a preferred embodiment, determining the ability of the Arginase II protein to bind to or interact with an Arginase II target molecule can be accomplished by determining the activity of the target molecule. For example, the activity of the target molecule can be determined by detecting induction of a cellular second messenger of the target (i.e., intracellular Ca²⁺, diacylglycerol, IP₃, and the like), detecting catalytic/enzymatic activity of the target an appropriate substrate, detecting the induction of a reporter gene (comprising a target-responsive regulatory element operatively linked to a nucleic acid encoding a detectable marker, e.g., luciferase), or detecting a target-regulated cellular response.

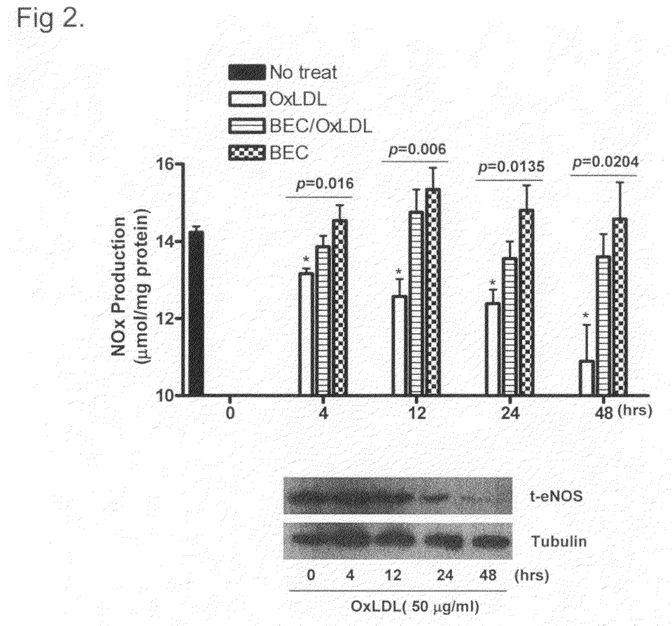

In yet another embodiment, an assay of the present invention is a cell-free assay in which an Arginase II protein or biologically active portion thereof is contacted with a test compound and the ability of the test compound to bind to the Arginase II protein or biologically active portion thereof is determined. Preferred biologically active portions of the Arginase II proteins to be used in assays of the present invention include fragments which participate in interactions with non-Arginase II molecules, e.g., fragments with high surface probability scores (see, for example, FIGS. 2 and 13). Binding of the test compound to the Arginase II protein can be determined either directly or indirectly as described above. In a preferred embodiment, the assay includes contacting the Arginase II protein or biologically active portion thereof with a known compound which binds Arginase II to form an assay mixture, contacting the assay mixture with a test compound, and determining the ability of the test compound to interact with an Arginase II protein, wherein determining the ability of the test compound to interact with an Arginase II protein comprises determining the ability of the test compound to preferentially bind to Arginase II or biologically active portion thereof as compared to the known compound.

In another embodiment, the assay is a cell-free assay in which an Arginase II protein or biologically active portion thereof is contacted with a test compound and the ability of the test compound to modulate (e.g., stimulate or inhibit) the activity of the Arginase II protein or biologically active portion thereof is determined. Determining the ability of the test compound to modulate the activity of an Arginase II protein can be accomplished, for example, by determining the ability of the Arginase II protein to bind to an Arginase II target molecule by one of the methods described above for determining direct binding. Determining the ability of the Arginase II protein to bind to an Arginase II target molecule can also be accomplished using a technology such as real-time Biomolecular Interaction Analysis (BIA). Sjolander, S, and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In an alternative embodiment, determining the ability of the test compound to modulate the activity of an Arginase II protein can be accomplished by determining the ability of the Arginase II protein to further modulate the activity of a downstream effector of an Arginase II target molecule. For example, the activity of the effector molecule on an appropriate target can be determined or the binding of the effector to an appropriate target can be determined as previously described.

In yet another embodiment, the cell-free assay involves contacting an Arginase II protein or biologically active portion thereof with a known compound which binds the Arginase II protein to form an assay mixture, contacting the assay mixture with a test compound, and determining the ability of the test compound to interact with the Arginase II protein, wherein determining the ability of the test compound to interact with the Arginase II protein comprises determining the ability of the Arginase II protein to preferentially bind to or modulate the activity of an Arginase II target molecule.

In more than one embodiment of the above assay methods of the present invention, it may be desirable to immobilize either Arginase II or its target molecule to facilitate separation of complexed from uncomplexed forms of one or both of the proteins, as well as to accommodate automation of the assay. Binding of a test compound to an Arginase II protein, or interaction of an Arginase II protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/Arginase II fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or Arginase II protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of Arginase II binding or activity determined using standard techniques.

Other techniques for immobilizing proteins on matrices can also be used in the screening assays of the invention. For example, either an Arginase II protein or an Arginase II target molecule can be immobilized utilizing conjugation of biotin and streptavidin. Biotinylated Arginase II protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with Arginase II protein or target molecules but which do not interfere with binding of the Arginase II protein to its target molecule can be derivatized to the wells of the plate, and unbound target or Arginase II protein trapped in the wells by antibody conjugation. Methods for detecting such complexes, in addition to those described above for the GST-immobilized complexes, include immunodetection of complexes using antibodies reactive with the Arginase II protein or target molecule, as well as enzyme-linked assays which rely on detecting an enzymatic activity associated with the Arginase II protein or target molecule.

In another embodiment, modulators of Arginase II expression are identified in a method wherein a cell is contacted with a candidate compound and the expression of Arginase II mRNA or protein in the cell is determined. The level of expression of Arginase II mRNA or protein in the presence of the candidate compound is compared to the level of expression of Arginase II mRNA or protein in the absence of the candidate compound. The candidate compound can then be identified as a modulator of Arginase II expression based on this comparison. For example, when expression of Arginase II mRNA or protein is greater (statistically significantly greater) in the presence of the candidate compound than in its absence, the candidate compound is identified as a stimulator of Arginase II mRNA or protein expression. Alternatively, when expression of Arginase II mRNA or protein is less (statistically significantly less) in the presence of the candidate compound than in its absence, the candidate compound is identified as an inhibitor of Arginase II mRNA or protein expression. The level of Arginase II mRNA or protein expression in the cells can be determined by methods described herein for detecting Arginase II mRNA or protein.

In yet another aspect of the invention, the Arginase II proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with Arginase II (“Arginase II-binding proteins” or “Arginase II-bp”) and are involved in Arginase II activity.

The two-hybrid system is based on the modular nature of most transcription factors, which consist of separable DNA-binding and activation domains. Briefly, the assay utilizes two different DNA constructs. In one construct, the gene that codes for an Arginase II protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an Arginase II-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the Arginase II protein.

Moreover, the ability of a test compound to inhibit the release of Arginase II from microtubules can be monitored as described in the examples. For example, an antibody specific for Arginase II can be used to visualize the location of Arginase II within a cell. Additionally, a second antibody specific for the microtubules can be visualized within the cell and the skilled artisan can determine if the Arginase II is bound to the microtubules. The ability of a compound to modulate the release of Argianse II from microtubules can therefore be monitored visually as described herein.

In another aspect, the invention pertains to a combination of two or more of the assays described herein. For example, a modulating agent can be identified using a cell-based or a cell free assay, and the ability of the agent to modulate the activity of an Arginase II protein can be confirmed in vivo, e.g., in an animal such as an animal model for atherogenesis.

This invention further pertains to novel agents identified by the above-described screening assays. Accordingly, it is within the scope of this invention to further use an agent identified as described herein in an appropriate animal model. For example, an agent identified as described herein (e.g., an Arginase II modulating agent, an antisense Arginase II nucleic acid molecule, an Arginase II-specific antibody, or an Arginase II-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

The present invention encompasses agents which modulate expression, activity or amount of free Arginase II. As used herein, the term “free Arginase II” is intended to mean the amount of Arginase II that is not bound to microtubules. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

The modulators of Arginase II of the invention may also be RNAi molecules. As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence or knockdown the expression of target genes, e.g., arginase II.

“RNAi molecule” or an “siRNA” refers to a nucleic acid that forms a double stranded RNA, which double stranded RNA has the ability to reduce or inhibit expression of a gene or target gene when the siRNA expressed in the same cell as the gene or target gene. “siRNA” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 nucleotides in length, e.g., 20, 21, 22, 23, 24, 25, 26, 27, 28, 29, or 30 nucleotides in length.

The modulators of Arginase II of the invention may also be antibodies. “Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells; Random combinations of the heavy and light chain gene products generate a large pool of antibodies with different antigenic specificity (see, e.g., Kuby, Immunology (3. sup.rd ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and coworkers (see, e.g., Jones et al., Nature 321:522-525 (1986); Riechmann et al. Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an, enzyme, toxin, hormone, growth factor, drug, etc.; or (b) the variable region, or a portion thereof, is altered, replaced or exchanged with a variable region having a different or altered antigen specificity.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies raised to Arginase II, polymorphic variants, alleles, orthologs, and conservatively modified variants, or splice variants, or portions thereof, can be selected to obtain only those polyclonal antibodies that are specifically immunoreactive with Arginase II and not with other proteins. This selection may be achieved by subtracting out antibodies that cross-react with other molecules. A variety of immunoassay formats may be used to select antibodies specifically immunoreactive with a particular protein.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The pharmaceutical compositions can be included in a kit, e.g., a container, pack, or dispenser, together with instructions for administration.

### Pharmaceutical Compositions

The modulators of Arginase II expression or activity described herein can be incorporated into pharmaceutical compositions suitable for administration. Such compositions typically comprise a small molecule, nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of a compound (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound can include a single treatment or, preferably, can include a series of treatments.

### Methods of Treatment

The present invention provides for both prophylactic and therapeutic methods of treating a subject at risk of (or susceptible to) a disorder or having a disorder associated with aberrant or unwanted Arginase II expression, regulation or activity, e.g. atherosclerotic disease. With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.)

Prophylactic Methods

In one aspect, the invention provides a method for preventing in a subject, a disease or condition associated with an aberrant or unwanted Arginase II expression or activity, e.g., atherosclerotic disease, by administering to the subject an agent which modulates Arginase II expression or Arginase II regulation. Subjects at risk for a disease which is caused or contributed to by aberrant or unwanted Arginase II expression or activity can be identified by, for example, any or a combination of diagnostic or prognostic assays as described herein. Administration of a prophylactic agent can occur prior to the manifestation of symptoms characteristic of the Arginase II aberrancy, such that a disease or disorder is prevented or, alternatively, delayed in its progression. Depending on the type of Arginase II aberrancy, for example, an Arginase II modulating compound can be used for treating the subject. The appropriate agent can be determined based on screening assays described herein.

Therapeutic Methods

Another aspect of the invention pertains to methods of modulating the level of free Arginase II, the expression of Arginase II or activity of Arginase II for therapeutic purposes, e.g., for the treatment of atherosclerotic disease. Accordingly, in an exemplary embodiment, the modulatory method of the invention involves contacting a cell with an agent that modulates Arginase II protein activity or the transcription or translation of Arginase II nucleic acid in a cell. An agent that modulates Arginase II protein activity can be an agent as described herein, such as a nucleic acid or a protein, an Arginase II antibody, an Arginase II agonist or antagonist, a peptidomimetic of an Arginase II agonist or antagonist, or other small molecule. Exemplary Arginase II inhibitors are known in the art, e.g., N-hydroxay-nor-L-arginine (Nor-NOHA) and S-(2-boronoethyl)-L-cysteine (BEC). In one embodiment, the agent inhibits the activity of Arginase II. Examples of such inhibitory agents include antisense Arginase II nucleic acid molecules, anti-Arginase II antibodies, and Arginase II inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression, activity, or disassociation from microtubules of an Arginase II protein or nucleic acid molecule. In one embodiment, the method involves administering an agent (e.g., an agent identified by a screening assay described herein), or combination of agents that modulates (e.g., upregulates or downregulates) Arginase II expression or activity. In another embodiment, the method involves administering an Arginase II inhibitory molecule, e.g., a small molecule, protein or nucleic acid molecule, as therapy to compensate for reduced, aberrant, or unwanted Arginase II expression or activity.

In particular embodiments, the therapeutic methods of the invention are useful for treating atherosclerotic disease.

In a further embodiment, the invention provides stents comprising an Arginase II inhibitory molecule. In further embodiments, the invention provides methods of treating a subject using the stents of the invention.

Diagnostic Methods

The instant invention demonstrates that Arginase II disassociates from microtubules in diseased cells. Accordingly, the instant invention provides diagnostic methods for determining if a subject has, or is as risk of developing, and atherosclerotic disease. In one embodiment, the levels of free, i.e., disassociated, Arginase II are determined and the levels are compared to the levels in a control sample, or to a normal level, wherein in increase in the amount of free Arginase II is characteristic of a subject having, or at risk of developing, atherosclerotic disease.

In another embodiment, the invention provides a method for characterizing a subject's risk profile of developing a future cardiovascular disorder associated with atherosclerotic disease comprising obtaining a level of free Arginase II in a sample and comparing the level of the free Arginase II to a predetermined free Arginase II value to establish a risk value, and characterizing the subject's risk profile of developing a future atherosclerotic disease based upon a combination of the risk value associated with increased levels of free Arginase II.

In a related embodiment, the instant invention also provides kits for the diagnosis of atherosclerotic disease. The kit comprises a reagent that specifically detects Arginase II and instructions for use. In a specific example the kit comprises a antibody specific for Arginse II and instructions for use. In a further embodiment, the kit comprises a second antibody specific for tubulin.

### Examples

It should be appreciated that the invention should not be construed to be limited to the examples that are now described; rather, the invention should be construed to include any and all applications provided herein and all equivalent variations within the skill of the ordinary artisan.

Materials and Methods

OxLDL, prepared by reaction with CuSO₄, was purchased from Intracel Co (Frederick, Md.). The remainder of the chemicals used in this study were obtained from Sigma Co.

Cell Culture

Human aortic endothelial cells (HAECs) were purchased from Cascade Biologics (Portland, Oreg.) and maintained in Medium M200 containing low serum growth supplement according to the supplier's protocol. Confluent HAECs were incubated with starvation medium (M200 plus only 0.5% fetal bovine serum) for 24 hr prior to the experimental protocols.

Arginase Activity Assay

Cell lysates were prepared with lysis buffer (50 mM Tris-HCl, pH7.5, 0.1 mM EDTA and protease inhibitors) by brief vigorous vortexing for 30 min and incubation and centrifuging for 20 min at 14,000 g at 4° C. The supernatants are used for arginase activity as described previously¹⁸. Arginase activity from aortic vessels was assayed as described above following homogenization in lysis buffer.

NO Measurement

NO was estimated as nitrate/nitrite (NOx) by Griess reaction after conversion of nitrate to nitrite by nitrate reductase, using the Nitric Oxide Assay Kit (Calbiochem). The concentration of NOx from cell lysates was expressed as nmol/mg proteins.

Transfection of Arginase II-SiRNA

Transient transfection of arginase-SiRNA (Santa Cruz Biotechnology) was performed with Oligofectamine reagent according to instructions provided by the supplier (Invitrogen). In brief, 8 μL of Oligofectamine was added to 17 μL Opti-MEM I reduced serum medium (Gibco), incubated for 5 minutes at room temperature, mixed with 180 μL Opti-MEM medium containing different amount of SiRNA, and further incubated for 15 minutes. The SiRNA-Oligofectamine complex was then overlaid on cells (each well of a 6 well culture dish). After incubation for 6 h, endothelial growth medium containing 3 times serum was added for making normal growth medium of 1× serum concentration. Transfected cells were then further incubated for 36 hr and starved for 24 hr prior to experiments and then stimulated with OxLDL for additional 6 hours.

Western Blot Analysis

Cells were lysed in SDS sample buffer (62.5 mM Tris, pH6.8, 2% SDS, and 10% Glycerol) and then sonicated for 5 s to reduce sample viscosity. Each sample was resolved by 10% SDS-PAGE, transferred to PVDF membrane (Bio-rad), analysed with antibodies according to the supplier's protocol, and visualized with peroxidase and an enhanced-chemiluminescence system (Pierce). Normalization was performed using the anti-β-tubulin antibody (BD bioscience, 1:1,000). Densitometric analysis of bands was performed with NIH ImageJ program.

Immunoprecipitation

Tubulin proteins were immunoprecipitated with its antibody (Sigma, rabbit) by using techniques modified from previous described²². Briefly, washed endothelial cells with PBS were incubated on ice with following cytoskeletal stabilizing solution for 10 min (1% Triton X-100, 100 mM NaCl, 300 mM sucrose, 3 mM MgCl2, 1 mM EGTA, 1.2 mM PMSF, 10 mM PIPES pH7.2, protease inhibitors). The detergent insoluble cytoskeletal fractions (remaining in the tissue culture dishes) were scraped in RIPA immunoprecipitation buffer and sonicated shortly. After the immunoprecipitating solution was subjected to centrifugation (12,000 g, 4° C., 15 min) and clarified by preincubating with protein A/G agarose beads, tubulin proteins were precipitated with anti-tubulin antibody by incubating overnight at 4° C. Western blot analysis was performed with anti-arginase II antibody (from Santa Cruz Biotechnol, goat).

qRT-PCR

Total RNA from Ox-LDL-stimulated HAEC was prepared using Trizol Reagent according to the supplier's protocol (Gibco). To exclude contamination with genomic DNA, total RNA was treated with RNase-free DNase (Roche). PCR reaction was performed in iCycler optical system (Bio-rad) using SYBR green PCR master mix. The reverse transcriptional PCR primers are as follows: Arginase I: Forward 5′-GGC AAG GTG GCA GAA GTC A-3′, Reverse 5′-TGG TTG TCA GTG GAG TGT TG-3′, size of PCR products=163 bp; Arginase II: Forward 5′-CTA TCA GCA CTG GAT CTT GTT G-3′, Reverse 5′-GGG AGT AGG AAG TTG GTC ATA G-3′, size of PCR products=156 bp. 18S rRNA gene (Forward 5′-CGG CGA CGA CCC ATT CGA AC-3′, Reverse 5′-GAA TCG AAC CCT GAT TCC CCG TC-3′, size of PCR product=99 bp) was amplified as a control.

Immunofluorescence Microscopy

HAECs were cultured on coverslips coated with 25 □g/ml human plasma fibronectin (Invitrogen). Cells were then fixed and permeabilized with 3% paraformaldehyde and 0.5% Triton X-100 in PBS for 2 minutes, followed by 20 minutes of 3% paraformaldehyde alone. Samples were prepared for immunofluorescence analysis by incubating with a rabbit polyclonal antisera against arginase II (Santa Cruz Biotechnology, 1:50) and a mouse monoclonal antibody against β-tubulin (BD Biosciences, 1:50) for 30 minutes at 37° C. They were then rinsed in tris-buffered saline and incubated with Cy5-conjugated anti-rabbit IgG and Cy3-conjugated anti-mouse IgG (Chemicon, Temecula, Calif.). Images were acquired using a Nikon TE-200 epifluorescence microscope (with a 60× objective, and collected using Openlab software (Improvision, Lexington, Mass.) and an internally cooled 12-bit CCD camera (CoolsnapHQ, Photometrics, Tucson, Ariz.).

Tubulin Depolymerization Assay

To further evaluate tubulin depolymerization by OxLDL stimulation, a simple method was performed as previously described by Giannakakou et al²³, Briefly, stimulated cells were washed twice with PBS and lysed at 37° C. for 5 min in the dark with 150 μl of hypotonic buffer (1 mM MgCl2, 2 mM EGTA, 0.5% NP-40, 2 mM PMSF, protease inhibitor (from Sigma), 20 mM Tris-HCl, pH6.8). Lysis was followed by a brief but vigorous vortexing, and the lysates were centrifuged at 14,000×g for 10 min at room temperature. The 150 μl supernatants containing soluble (cytosolic) tubulin were transferred to fresh tubes. The pellets containing polymerized (cytoskeletal) tubulin were resuspended in 150 μl of hypotonic buffer and centrifuged again as above. Both the cytosolic and the polymerized fractions were used for both arginase activity assays and western blot analysis.

Statistics

All data are reported as mean±SEM. Each graph represents cumulative data from between 3-5 independent experiments. Each experimental assay was performed in triplicate. Statistical significance was determined by one way ANOVA with a post hoc test (Graphpad Prism 4 software).

Results

OxLDL Stimulation Increases Arginase Activity in HAEC.

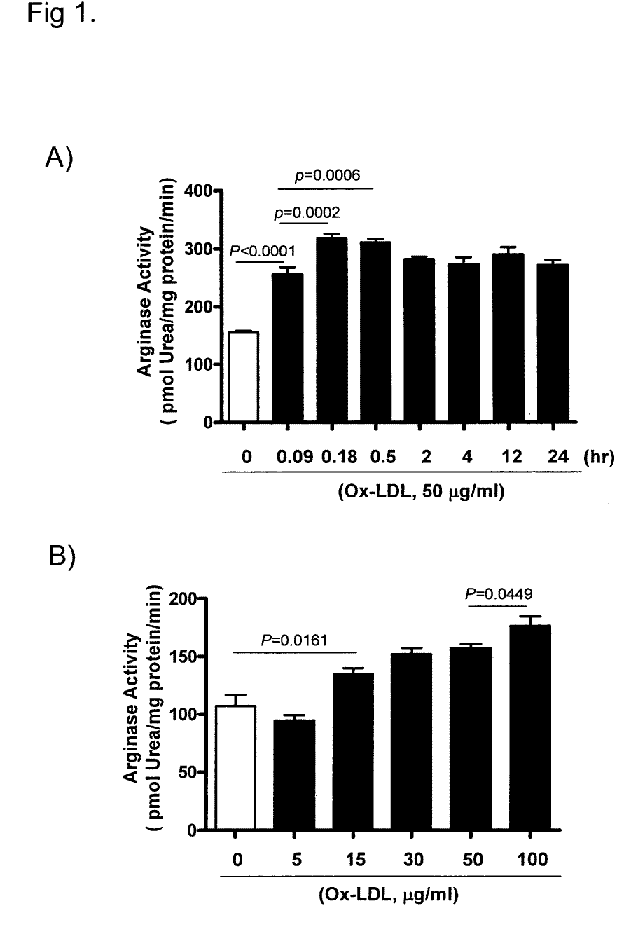

In order to determine whether OxLDL increased the activity of arginase in cultured human aortic endothelial cells the following experiments were preformed. In HAEC's, Ox-LDL (50 μg/ml) stimulation induced a time-dependent increase in arginase enzyme activity (FIG. 1a). Arginase activity was increased as early as 5 minutes (1.6-fold increase versus control, n=9, p<0.0001) and persisted for 48 hours. The maximal arginase activity was observed 10 minutes after OxLDL-stimulation (2.0-fold increase versus control, n=9, p<0.0001). In addition, the dose-dependent nature of the OxLDL effect on arginase activity was measured in endothelial cells at 10 minutes. The enzyme activity gradually increased in a dose-dependent manner (FIG. 1b) with a maximal effect at 100 μg/ml of OxLDL (1.64-fold versus control, p<0.0001). Given that the submaximal responses occurred at 50 μg/ml (OxLDL 50 μg/ml VS. 100 μg/ml, p=0.0449), this concentration was used for all subsequent experiments. The dose of 25-50 μg/ml is within the order of magnitude one would observe clinically²⁴.

OxLDL Stimulation Reciprocally Decreases NO Production

Based on recent data indicating that Arginase reciprocally regulates NOS activity by limiting L-arginine bioavailability, it was tested whether an OxLDL-induced increase in arginase activity was associated with a decrease in NOx measurement. Since it has been previously demonstrated that eNOS expression is decreased in endothelial cells exposed to OxLDL, both total eNOS abundance and NOx at different time intervals following OxLDL stimulation was measured. As demonstrated in FIG. 2, OxLDL stimulation results in a time dependent decrease in NOx production starting as early as our first time point (4 hrs) and continuing to decrease further, reaching a minimum level of 34% of baseline (OxLDL vs control, n=6, p=0.0018) at 48 hours. eNOS abundance remains constant until between 12 and 24 hrs when expression levels begin to decline. Arginase inhibitions with S-(2-boronoethyl)-L-cysteine (BEC, 10 μmol/L) prevents OxLDL-dependent decreases is NO at all tirne points starting as early as 4 hrs (102%, OxLDL+BEC vs. control=14.58±0.94 vs 14.24±0.95, n=6). Thus OxLDL dependent decreases in NOx production occur before declines in eNOS abundance. Furthermore arginase inhibition prevents the OxLDL dependent decrease in NOx production at all time points despite a decrease in eNOS expression. BEC alone had no effect on HAEC NOx production.

Arginase II is the Key Isoform Regulating Arginase Activity and NO Production in HAECs

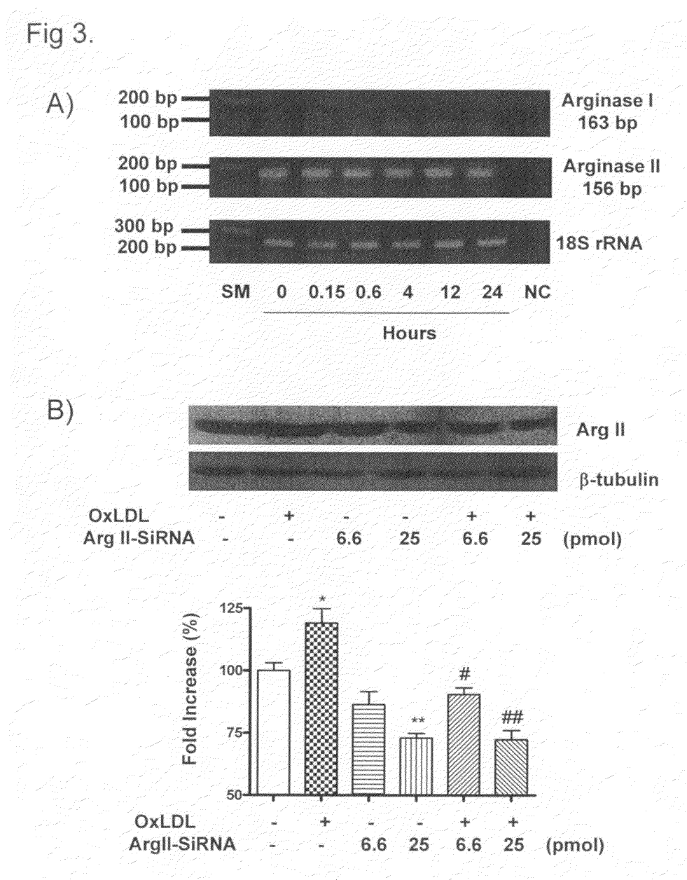

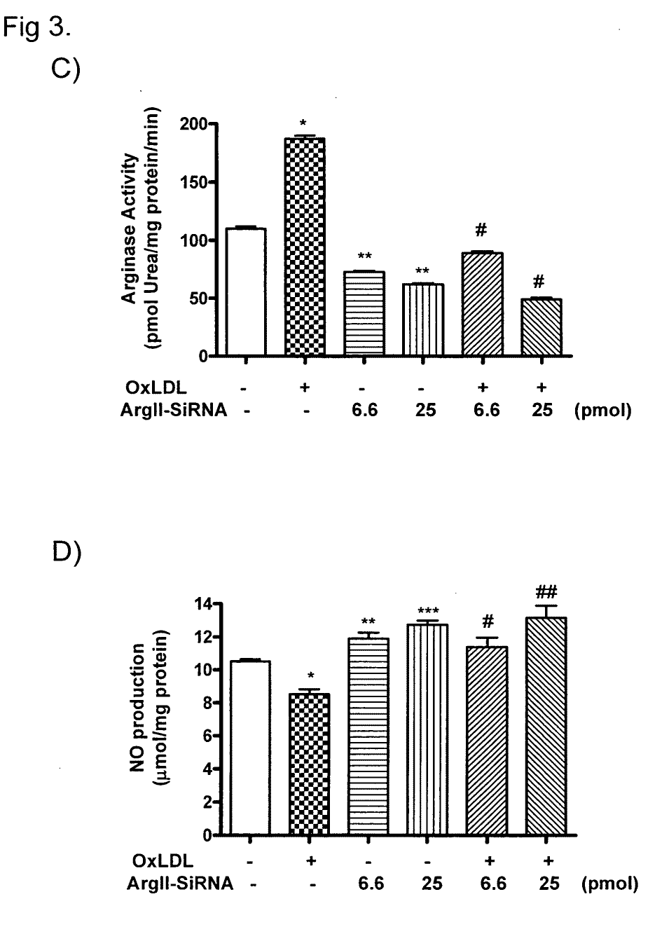

Since it is well known that 2 isoforms of arginase exist^{10,11} and that both isoforms have been demonstrated to regulate NOS activity, it was determine which isoform of the enzyme is expressed in HAEC and which therefore may be responsible for reciprocal regulation of NOS. RT-PCR was performed with specific primer sets for arginase I and II. As demonstrated in FIG. 3a, the expression of arginase II was identified by its RT-PCR product in both nonstimulated and OxLDL stimulated cells. Arginase I expression was not detected in either stimulated or non-stimulated HAECs. Given that only arginase II appears to be expressed in HAEC's. The functional role of this isoform was investigated. Because of the lack of isoform-specific arginase inhibitors, a small interference RNA (SiRNA) technique to perform loss of function experiments was used. SiRNA targeted to arginase II was transfected into HAEC by Oligofectamine reagent. Incubation of SiRNA for 36 hours significantly decreased arginase II protein abundance (FIG. 3b) to 72% (100±5.32 vs 72.98±3.25, control vs SiRNA (25 μM), p=0.0017) of baseline. The decreased protein abundance was associated with a proportional decrease in arginase enzyme activity. As demonstrated in FIG. 3c, SiRNA at the concentration of 6.6 μmol/L decreased enzyme activity from 193.2±26.7 (OxLDL stimulated group) to 93.2 pmol of Urea per mg protein per min. The arginase activity was further decreased (57.8±7.7 μmol Urea/mg protein/min) by increasing the concentration of arginase II-targeted SiRNA to 25 pmol/L (p=0.0025). Furthermore NOS activity was increased from 10.6±0.11 to 13.7±1.18 at 25 μmol/L of SiRNA arginase II. This represents a 1.57-fold increase compared to the untreated control (8.7±0.79 vs 13.7±1.18, n=6, p=0.0002). Thus, arginase II appears to be the predominant isoform responsible for reciprocal regulation of NOS in HAEC's. Furthermore, knockdown of arginase II can prevent OxLDL-induced increases in HAEC arginase activity.

Transcriptional Induction and Translational Activation of Arginase II by OxLDL

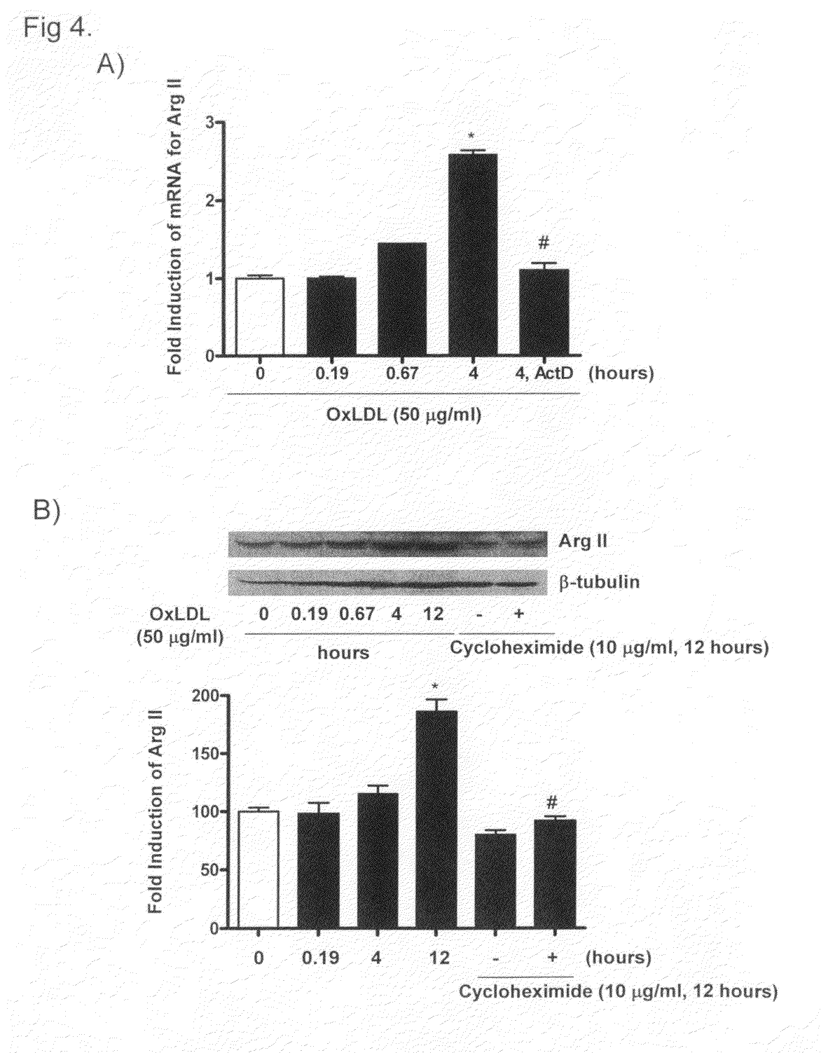

Given the time-dependent increase in arginase II activity following OxLDL stimulation the molecular mechanism underlying this phenomenon was evaluated. It was first determined whether OxLDL increased the available pool of arginase II at a transcriptional level. Quantitative PCR was performed at different time intervals. As seen in FIG. 4, there was a significant increase in arginase II expression at 4 hrs (˜2.5-fold, p<0.0001). This was completely blocked by the transcriptional inhibitor, actinomysin D (10 μg/ml). The induced mRNA II was also translated to arginase II protein after OxLDL stimulation for 4 hours and significantly increased after 12-hour stimulation (1.8-fold, p=0.0015). This was completely blocked by co-incubation of cells with the translational inhibitor, cycloheximide (10 μM.

It is important to note, however, that increases in arginase activity as early as 5 min following OxLDL stimulation cannot be accounted for by alterations in transcription or translation. This suggests a post-translational mechanism for the early activation of arginase II following OxLDL stimulation of HAEC.

Dissociation of Arginase II from Microtubules is a Key Mechanism of Arginase Activation

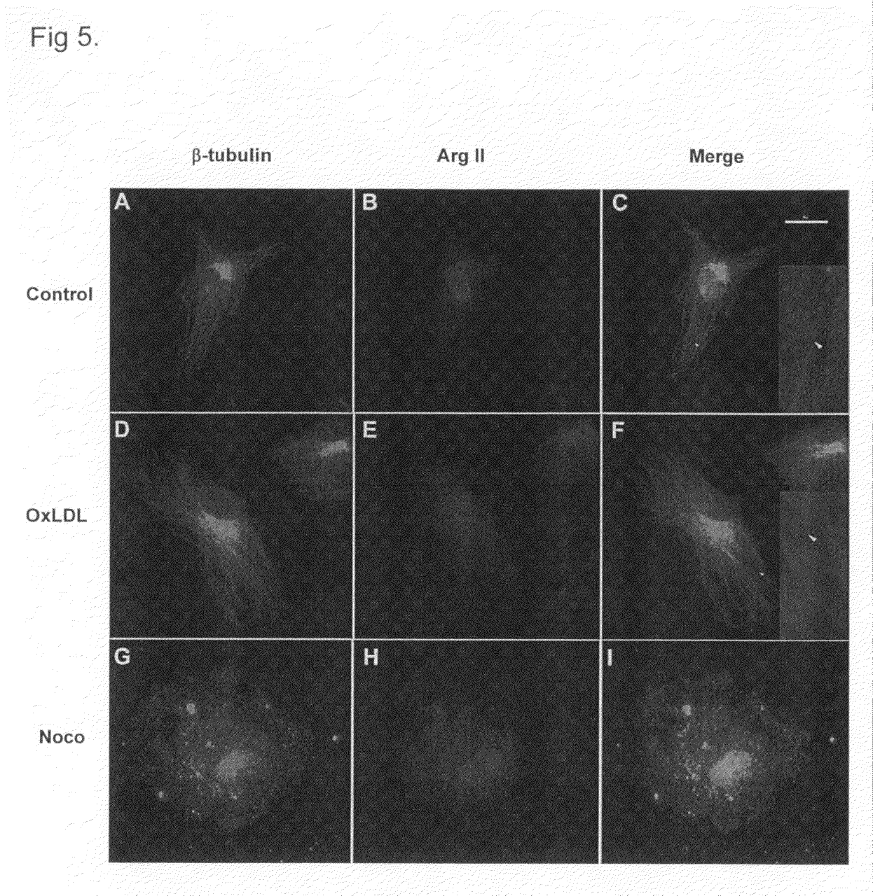

The rapid activation of arginase II following OxLDL stimulation indicates that changes in the availability or activation state of the enzyme may be occurring. Immunofluorescence imaging was used to map the topography of arginase II with respect to both actin microfilaments and microtubules in HAEC. Imaging revealed no correspondence between arginase II and the actin cytoskeleton, but a striking colocalization with microtubules (stained with an anti-β-tubulin antibody) was seen (FIG. 5). This association of arginase II with microtubules was disrupted by OxLDL. To further investigate the dependence of arginase II distribution upon the structure of microtubular networks, microtubules were depolymerized with nocodazole (50 μM). Microtubule depolymerization caused a dramatic redistribution of arginase II to a diffuse cytosolic pattern. Thus, the dissociation of arginase from the microtubules represents a novel molecular activation mechanism.

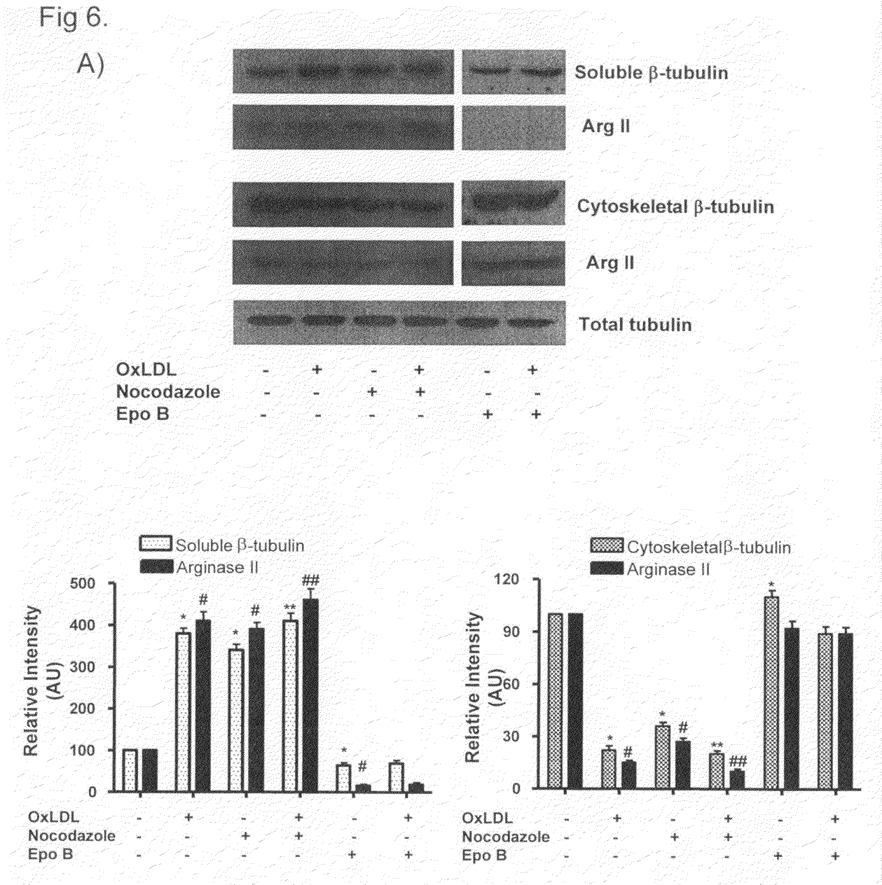

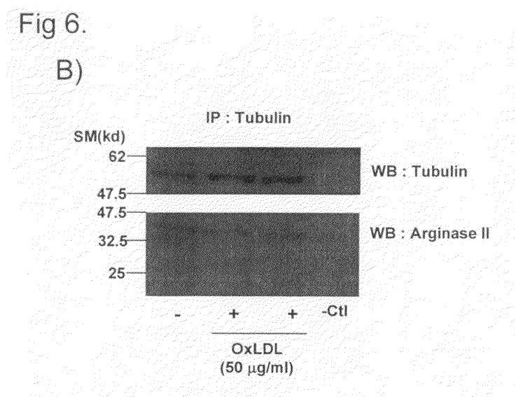

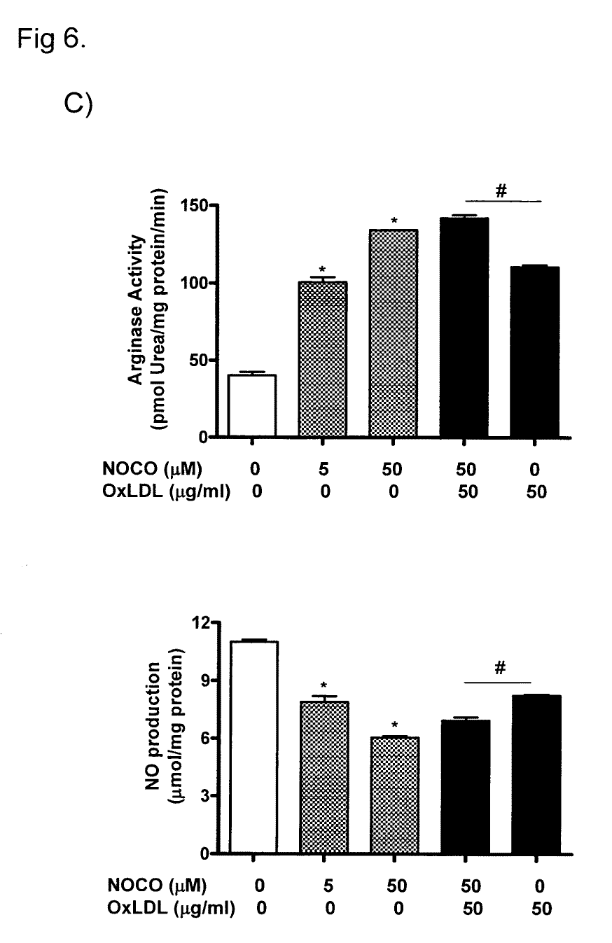

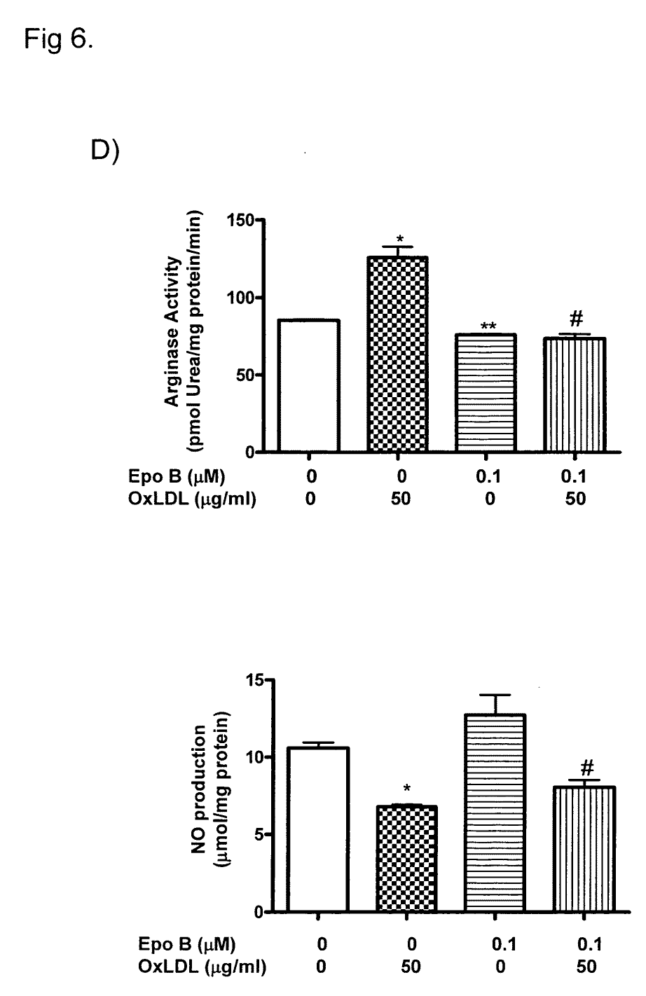

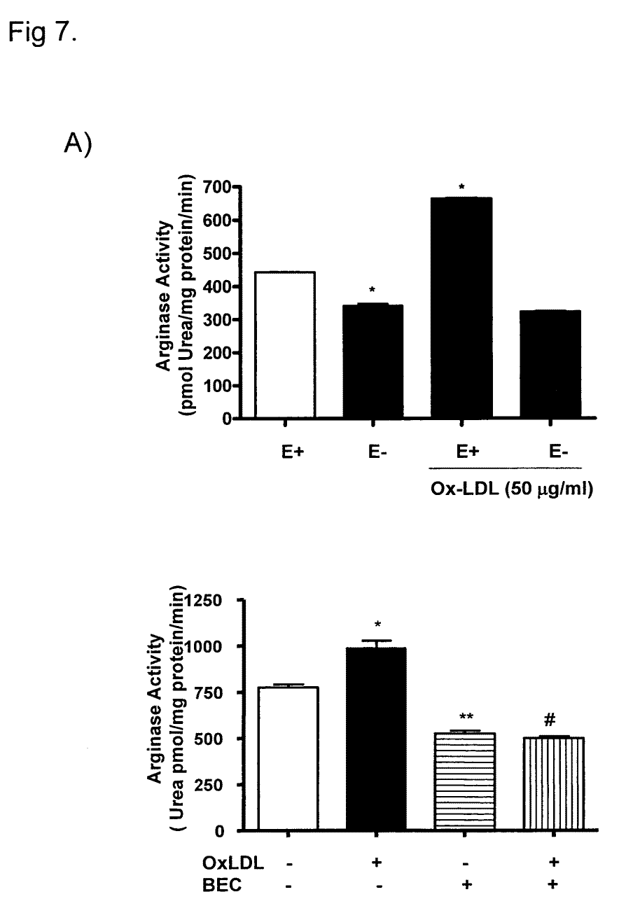

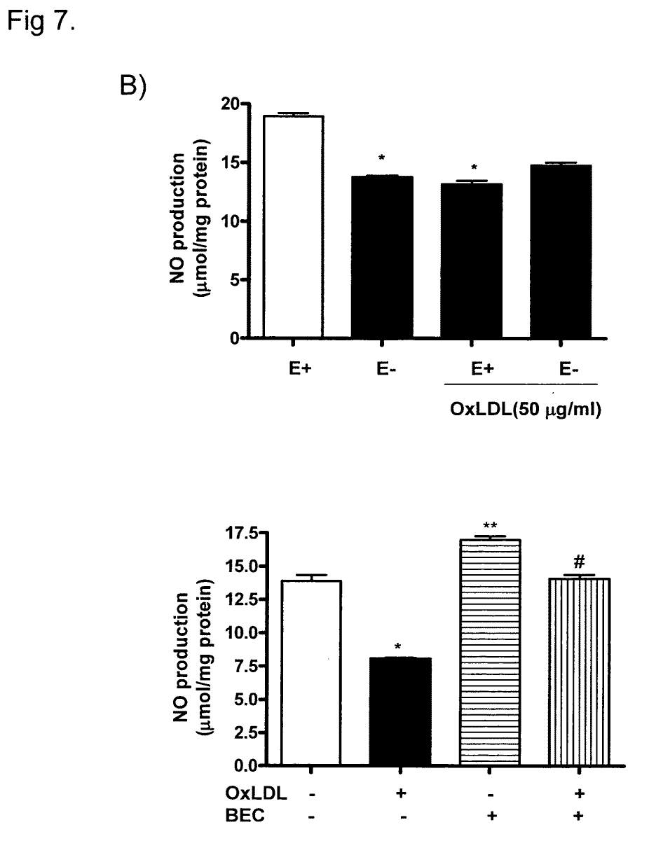

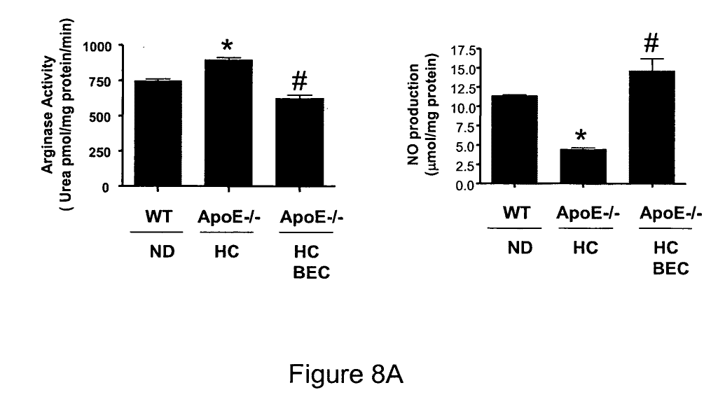

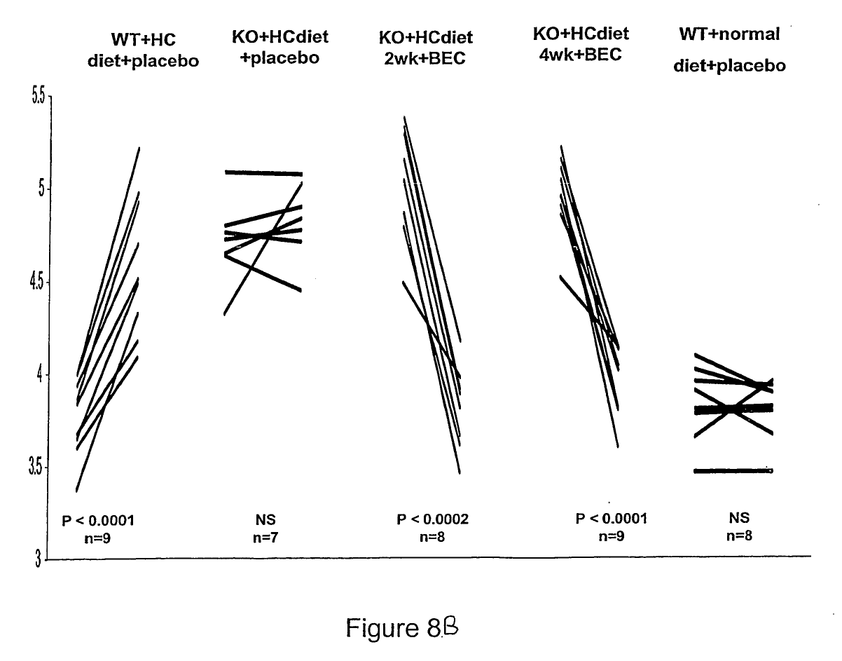

In order to quantitate the dependence of OxLDL-mediated increases in arginase activity on release from microtubular association, tubulin depolymerization assays were performed. Briefly, cell lysates were separated into an insoluble, polymerized tubulin fraction and a soluble, depolymerized tubulin fraction. The total amounts of tubulin, arginase II, and arginase activity were measured in each fraction. OxLDL treatment led to a significant redistribution of tubulin from the insoluble fraction to the soluble fraction within 5 minutes after treatment (FIG. 6A). This tubulin redistribution was also accompanied by a concomitant redistribution of arginase II to the soluble fraction and an increase in arginase activity. Similar results were seen using nocodazole as a positive control. Thus, arginase II was found primarily in association with the microtubule cytoskeleton fraction in untreated cells, where its activity appeared to be constrained, but it was redistributed to the soluble (cytosolic) fraction and activated upon OxLDL stimulation. Thus OxLDL appears to increase arginase activity by inducing microtubule depolymerization and release of the enzyme into the cytosol. To further confirm whether an interaction of arginase II and tubulin exists, cell lysates solubilized in RIPA buffer after tubulin stabilization were adjusted to immunoprecipitation with anti-tubulin specific antibody (FIG. 6B). As predicted, arginase II was co-immunoprecipitated with tubulin in immunoblot analysis, but not in negative control without tubulin antibody.

Indeed, nocodazole treatment increased arginase activity in a dose-dependent manner (40.4±7.63, 100.7±11.17, and 134.3±0.28 μmol Urea/mg protein/min respectively, in control conditions and at 5 μmol/L, and 50 μmol/L nocodazole, p<0.0001). Nocodazole in combination with Ox-LDL increased arginase to a level (141.9±7.07) that was statistically different from Ox-LDL alone, suggesting that they may act by a different mechanism (FIG. 6C top). Nocodazole treatment also led to a reciprocal decrease in NOS measurement (FIG. 6C bottom). Next, epothilone B was used to stabilize the microtubules by halting depolymerization. Epothilone B (0.1 μmol/L) markedly inhibited OxLDL-induced arginase activation (FIG. 6D top, 73.55±2.82 vs. 125.65±6.85, OxLDL+epothilone B vs. OxLDL, n=6, p=0.0015). Epothilone B alone resulted in a small but not statistically significant increase in basal endothelial cell NOx. Epothilone B while completely blocking OxLDL dependent arginase activity, attenuated but did not completely block OxLDL mediated decreases in endothelial NO production (6.79±0.14 vs. 8.05±0.47, OxLDL vs. OxLDL+epothilone B, n=6, p=0.0293). This suggests that prevention of dissociation of arginase by OxLDL is unlikely the sole mechanism by which epothilone B may modulate endothelial NO signaling.

Thus, both OxLDL treatment and nocodazole induced microtubule depolymerization results in increased arginase II activity and decreased NOS activity, while epothilone B-dependent stabilization of the microtubular structure prevents OxLDL-dependent activation of arginase II and attenuates the decrease in NO production. This lends further support to our hypothesis that OxLDL-dependent arginase II activation is mediated by its association with microtubules.

Arginase Activation and Reciprocal NO Decrease in OxLDL-Treated Rat Aorta

Preincubation of rat aortic rings with OxLDL (16 hrs) resulted in a significant increase in arginase activity in aortic rings in which the endothelium remained intact (E+, n=5), but not in rings in which the endothelium had been denuded (E−, n=4). This is consistent with our previous observations confirming that arginase is confined primarily to the endothelium in vascular tissue¹⁸. Furthermore, increased arginase activity was associated with a reciprocal decrease in NO production in E+ rings. On the other hand, pre-incubation of the E+rings with the arginase-specific inhibitor (S)-(2-Boronoethyl)-L-cysteine (BEC), decreased arginase activity and increased vascular NO production following OxLDL treatment.

Endothelial Expression of Arginase

It is increasingly recognized that arginase is constitutively expressed in endothelial cells of multiple vascular beds^{11,26-28}. It has recently been demonstrated that both isoforms are constitutively expressed in human umbilical vein endothelial cells where they regulate progression through the cell cycle (inhibition of arginase leads to growth inhibition)¹⁷. The predominant isoform in this cell population appears to be arginase I. In contrast, in a porcine coronary artery model, Zhang et al²⁹ have shown that the arginase I isoform is mainly responsible for limiting endothelial-dependent relaxation. Moreover, bovine pulmonary EC's express both arginase I and II that can be upregulated by cytokines, and arginase inhibition in these cells accentuates NO release⁹. We have previously demonstrated constitutive expression of both arginase I and II in rat aortic endothelium where arginase I is the predominant isoform¹⁸. Our ongoing studies in the rat aorta (using antisense technology) have demonstrated that arginase I is the isoform responsible for reciprocal regulation of NOS in these endothelia and that its function and abundance are increased with aging⁸.

qRT-PCR and western blot data presented herein demonstrate that arginase II is the predominant isoform expressed in the human aortic endothelial cells. This was further supported by the siRNA experiment in which only arginase II-specific SiRNA modulated arginase activity and reciprocally enhanced NOS activity. SiRNA specific for arginase II decreased protein abundance in a dose-dependent manner and dramatically inhibited OxLDL-dependent arginase activation.

Post-Translational Activation of Arginase

Immunofluorescence data and tubulin polymerization assays, demonstrate that OxLDL induces the dissociation of arginase II from the microtubule cytoskeleton. Nocodazole causes microtubule depolymerization by quadrupling the rate of GTP hydrolysis on the tubulin dimer³⁰, and dramatically disrupts microtubule structure and function (see FIG. 5G). Nocodazole also induces the activation of arginase. In contrast, OxLDL induces a less dramatic degree of microtubule destabilization, but still causes net depolymerization of microtubules and arginase activation. Additionally, epothilone B, a microtubule-stabilizing agent, prevented both OxLDL-dependent microtubule depolymerization and arginase activation. Taken together, these data suggest that OxLDL activates arginase via a novel mechanism involving disengagement from the microtubule cytoskeleton in a manner that does not lead to complete disruption of microtubule infrastructure. The data indicate that microtubule-mediated sequestration may regulate the activity of arginase II in HAEC. Recent reports describe a cellular strategy for post-translational regulation of iNOS in several different cell types that may lend insight into the findings described in our paper³². In this work, the down-regulation of iNOS activity is shown to occur via incorporation of the enzyme into aggresomes in a dynein- and dynactin-dependent process that is abrogated by microtubule disruption with nocodazole. Activation of iNOS is accompanied by release from these aggresomes, which are juxtanuclear and are associated with both the microtubule organizing center and mitochondria.

Reciprocal Regulation of NOS

The data presented in this Example demonstrate that arginase activation by OxLDL reciprocally downregulates NO production in a time-dependent manner. This decrease in NO is completely inhibited by the arginase inhibitor BEC despite a time-dependent decrease in total eNOS abundance, suggesting that the decrease in basal NO in endothelial cells by OxLDL is predominantly dependent on arginase up-regulation. This finding is consistent with the idea that OxLDL may result in eNOS uncoupling in which case instead of producing NO, O2- is produced by the enzyme. This results from electrons flowing from the reductase domain in the heme to molecular oxygen rather than L-Arginine. There are a number of circumstances in which this may occur, specifically BH4 cofactor deficiency as well as a relative deficiency of L-arginine³³. It is our primary thesis that the upregulation of arginase contributes to this mechanism and that the inhibition of arginase therefore restores NO production. Thus it is the availability of substrate co-factors and local eNOS microdomain concentrations of L-arginine rather than the expression level/abundance of the eNOS enzyme that is critical in NO production. Furthermore, Decrease in NO production and its restoration with arginase inhibition (BEC) precedes the decrease in NOS expression supporting this alternate contributory mechanism whereby OxLDL impairs nitroso-redox balance in the EC's. Other proposed mechanisms for OxLDL-induced decrements in NO availability include the upregulation of caveolin-1 expression followed by eNOS sequestration, increased ROS production with subsequent decreased NO bioavailability³⁴ and decreased eNOS activity via inhibition of PKC-α-mediated phosphorylation of eNOS threonine 495³⁵. An additional factor in the reciprocal regulation of eNOS activity by arginase may be subcellular compartmentalization. NOS-3 is known to bind to the scaffolding protein caveolin-1 which serves as the structural backbone of the plasma membrane invaginations known as caveolae which have several well-described signal transduction functions³⁶. Caveolae are shuttled along microtubules from the cell periphery and plasma membrane-proximal sites to perinuclear sites neighboring the microtubule organizing center and nocodazole-induced microtubule depolymerization is associated with a dramatic increase in the membrane-associated pool of caveolin-1³⁷⁻³⁹. This topography of caveolar distribution, the proximity of caveolar networks to the microtubule cytoskeleton, and the direct dependence of caveolar trafficking upon microtubular function are all well described in endothelial cells^{38,40}. NOS-3, has been shown to be regulated by binding to caveolin-1 and sequestration within caveolae, and both of these processes constrain NOS-3 activity³⁶. NOS-3 activation is known be mediated by a complex of signaling elements clustered at caveolae in tight spatial arrays³⁶. These regulate Ca2+/calmodulin binding and subsequent dissociation of NOS-3 from caveolin-1. These events may occur preferentially at the cell surface. Microtubule-dependent NOS-3 trafficking may therefore regulate NOS-3 activation.

Additionally, the release of arginase from the microtubules may modulate NOS-3 activity through competition for L-arginine substrate. Dissociation of arginase from microtubule-dependent mechanisms may bring it into proximity of L-arginine pools that are shared by NOS and were not available to it when bound to tubulin. This phenomenon may indeed explain the time course of decreased NO production. While arginase activity is increased rapidly it is only after 4 hrs that NO production begins to decrease after 4 hours. This may represent a time-dependent depletion of the NOS-accessible L-arginine pool by arginase to a point at which L-arginine become substrate limiting.

Transcriptional Regulation of Arginase

The data presented herein clearly demonstrates two temporally distinct mechanisms responsible for the activation of arginase. One involves dissociation from the microtubules and the other involves an increase in mRNA transcription leading to an increase in protein levels. Arginase I expression has been shown to be up-regulated in wound-derived fibroblasts and rat aortic smooth muscle cells following stimulation with TGF-β and IL-4, IL-4 and IL-13. Furthermore arginase I expression appears to be regulated by the transcription factors CTF/NF-1, Sp1 and C/EBP⁴¹. With regard to arginase II, LPS stimulation induces its expression in rat aortic endothelial cells and macrophages but the involved transcriptional factors remain to be elucidated²⁷. Thus in pathophysiologic scenarios such as sepsis, arginase I and arginase II are co-induced with iNOS following LPS administration, leading to speculation that arginase I may limit sustained overproduction of NOS.

Rescue of Vascular Endothelial NO Production:

Incubation of OxLDL with endothelialized rat aortic rings resulted in a significant impairment in endothelial NO production This is consistent with a plethora of in vivo and in vitro data in human⁴² and animals⁴³ demonstrating that impairment of endothelial function and NO production is a hallmark of OxLDL-mediated atherosclerotic disease. Therefore, the data presented in this example indicate that arginase is a therapeutic target for OxLDL dependent endothelial dysfunction.

### REFERENCES

1. Khan B V, Parthasarathy S S, Alexander R W, Medford R M. Modified low density lipoprotein and its constituents augment cytokine-activated vascular cell adhesion molecule-1 gene expression in human vascular endothelial cells. J Clin Invest. 1995; 95:1262-70.
2. Liao L, Starzyk R M, Granger D N. Molecular determinants of oxidized low-density lipoprotein-induced leukocyte adhesion and microvascular dysfunction. Arterioscler Thromb Vasc Biol. 1997; 17:437-44.
3. Galle J, Bengen J, Schollmeyer P. Wanner C. Impairment of endothelium-dependent dilation in rabbit renal arteries by oxidized lipoprotein(a). Role of oxygen-derived radicals. Circulation. 1995; 92:1582-9.
4. Dimmeler S, Haendeler J, Galle J, Zeiher A M. Oxidized low-density lipoprotein induces apoptosis of human endothelial cells by activation of CPP32-like proteases. A mechanistic clue to the ‘response to injury’ hypothesis. Circulation. 1997; 95:1760-3.
5. Harada-Shiba M, Kinoshita M, Kamido H, Shimokado K. Oxidized low density lipoprotein induces apoptosis in cultured human umbilical vein endothelial cells by common and unique mechanisms. J Biol. Chem. 1998; 273:9681-7.
6. Kugiyama K, Kerns S A, Morriset J D, Roberts R, Henry P D. Impairment of endothelium-dependent arterial relaxation by lysolecithin in modified low-density lipoproteins. Nature. 1990; 344:160-2.
7. Simon B C, Cunningham L D, Cohen R A. Oxidized low density lipoproteins cause contraction and inhibit endothelium-dependent relaxation in the pig coronary artery. J Clin Invest. 1990; 86:75-9.
8. Berkowitz D E, White R, Li D, Minhas K M, Cemetich A, Kim S, Burke S, Shoukas A A, Nyhan D, Champion H C, Hare J M. Arginase reciprocally regulates nitric oxide synthase activity and contributes to endothelial dysfunction in aging blood vessels. Circulation. 2003; 108:2000-6.
9. Chicoine L G, Paffett M L, Young T L, Nelin L D. Arginase inhibition increases nitric oxide production in bovine pulmonary arterial endothelial cells. Am J Physiol Lung Cell Mol. Physiol. 2004; 287:L60-8.
10. Haraguchi Y, Takiguchi M, Amaya Y, Kawamoto S, Matsuda I, Mori M. Molecular cloning and nucleotide sequence of cDNA for human liver arginase. Proc Natl Acad Sci USA. 1987; 84:412-5.
11. Morris S M, Jr., Bhamidipati D, Kepka-Lenhart D. Human type II arginase: sequence analysis and tissue-specific expression. Gene. 1997; 193:157-61.
12. Chang C I, Zoghi B, Liao J C, Kuo L. The involvement of tyrosine kinases, cyclic AMP/protein kinase A, and p38 mitogen-activated protein kinase in IL-13-mediated arginase I induction in macrophages: its implications in IL-13-inhibited nitric oxide production. J. Immunol. 2000; 165:2134-41.
13. Koga T, Koshiyama Y, Gotoh T, Yonemura N, Hirata A, Tanihara H, Negi A, Mori M. Coinduction of nitric oxide synthase and arginine metabolic enzymes in endotoxin-induced uveitis rats. Exp Eye Res. 2002; 75:659-67.
14. Louis C A, Reichner J S, Henry W L, Jr., Mastrofrancesco B, Gotoh T, Mori M, Albina J E. Distinct arginase isoforms expressed in primary and transformed macrophages: regulation by oxygen tension. Am J. Physiol. 1998; 274:R775-82.
15. Que L G, Kantrow S P, Jenkinson C P, Piantadosi C A, Huang Y C. Induction of arginase isoforms in the lung during hyperoxia. Am J. Physiol. 1998; 275:L96-102.
16. Sonoki T, Nagasaki A, Gotoh T, Takiguchi M, Takeya M, Matsuzaki H, Mori M. Coinduction of nitric-oxide synthase and arginase I in cultured rat peritoneal macrophages and rat tissues in vivo by lipopolysaccharide. J Biol. Chem. 1997; 272:3689-93.
17. Li H, Meininger C J, Hawker J R, Jr., Haynes T E, Kepka-Lenhart D, Mistry S K, Morris S M, Jr., Wu G. Regulatory role of arginase I and II in nitric oxide, polyamine, and proline syntheses in endothelial cells. Am J Physiol Endocrinol Metab. 2001; 280:E75-82.
18. White A R, Ryoo S, Li D, Champion H C, Steppan J, Wang D, Nyhan D, Shoukas A A, Hare J M, Berkowitz D E. Knockdown of arginase I restores NO signaling in the vasculature of old rats. Hypertension. 2006; 47:245-51.
19. Morris S M, Jr., Kepka-Lenhart D, Chen L C. Differential regulation of arginases and inducible nitric oxide synthase in murine macrophage cells. Am J. Physiol. 1998; 275:E740-7.
20. Bivalacqua T J, Hellstrom W J, Kadowitz P J, Champion H C. Increased expression of arginase II in human diabetic corpus cavemosum: in diabetic-associated erectile dysfunction. Biochem Biophys Res Commun. 2001; 283:923-7.
21. Ming X F, Barandier C, Viswambharan H, Kwak B R, Mach F, Mazzolai L, Hayoz D, Ruffieux J, Rusconi S, Montani J P, Yang Z. Thrombin stimulates human endothelial arginase enzymatic activity via RhoA/ROCK pathway: implications for atherosclerotic endothelial dysfunction. Circulation. 2004; 110:3708-14.
22. Romer L H, McLean N V, Yan H C, Daise M, Sun J, DeLisser H M. IFN-gamma and TNF-alpha induce redistribution of PECAM-1 (CD31) on human endothelial cells. J. Immunol. 1995; 154:6582-92.
23. Giannakakou P, Sackett D L, Kang Y K, Zhan Z, Buters J T, Fojo T, Poruchynsky M S. Paclitaxel-resistant human ovarian cancer cells have mutant beta-tubulins that exhibit impaired paclitaxel-driven polymerization. J Biol. Chem. 1997; 272:17118-25.
24. Holvoet P, Kritchevsky S B, Tracy R P, Mertens A, Rubin S M, Butler J, Goodpaster B, Harris T B. The metabolic syndrome, circulating oxidized LDL, and risk of myocardial infarction in well-functioning elderly people in the health, aging, and body composition cohort. Diabetes. 2004; 53:1068-73.
25. Byrd C A, Bormnann W, Erdjument-Bromage H, Tempst P, Pavletich N, Rosen N, Nathan C F, Ding A. Heat shock protein 90 mediates macrophage activation by Taxol and bacterial lipopolysaccharide. Proc Natl Acad Sci USA. 1999; 96:5645-50.
26. Bachetti T, Comini L, Francolini G, Bastianon D, Valetti B, Cadei M, Grigolato P, Suzuki H, Finazzi D, Albertini A, Curello S, Ferrari R. Arginase pathway in human endothelial cells in pathophysiological conditions. J Mol Cell Cardiol. 2004; 37:515-23.
27. Buga G M, Singh R, Pervin S, Rogers N E, Schmitz D A, Jenkinson C P, Cederbaum S D, Ignarro L J. Arginase activity in endothelial cells: inhibition by NG-hydroxy-L-arginine during high-output NO production. Am J. Physiol. 1996; 271:H1988-98.
28. Xu W, Kaneko F T, Zheng S, Comhair S A, Janocha A J, Goggans T, Thunnissen F B, Farver C, Hazen S L, Jennings C, Dweik R A, Arroliga A C, Erzurum S C. Increased arginase II and decreased NO synthesis in endothelial cells of patients with pulmonary arterial hypertension. Faseb J. 2004; 18:1746-8.
29. Zhang C, Hein T W, Wang W, Chang C I, Kuo L. Constitutive expression of arginase in microvascular endothelial cells counteracts nitric oxide-mediated vasodilatory function. Faseb J. 2001; 15:1264-6.
30. Mejillano M R, Shivanna B D, Himes R H. Studies on the nocodazole-induced GTPase activity of tubulin. Arch Biochem Biophys. 1996; 336:130-8.
31. Gundersen G G, Cook T A. Microtubules and signal transduction. Curr Opin Cell Biol. 1999; 11:81-94.
32. Kolodziejska K E, Burns A R, Moore R H, Stenoien D L, Eissa N T. Regulation of inducible nitric oxide synthase by aggresome formation. Proc Natl Acad Sci USA. 2005; 102:4854-9.
33. Sullivan J C, Pollock J S. Coupled and uncoupled NOS: separate but equal? Uncoupled NOS in endothelial cells is a critical pathway for intracellular signaling. Circ Res. 2006; 98:717-9.
34. Harrison D G. Cellular and molecular mechanisms of endothelial cell dysfunction. J Clin Invest. 1997; 100:2153-7.
35. Fleming I, Mohamed A, Galle J, Turchanowa L, Brandes R P, Fisslthaler B, Busse R. Oxidized low-density lipoprotein increases superoxide production by endothelial nitric oxide synthase by inhibiting PKCalpha. Cardiovasc Res. 2005; 65:897-906.
36. Marx J. Caveolae: a once-elusive structure gets some respect. Science. 2001; 294:1862-5.
37. Parton R G, Joggerst B, Simons K. Regulated internalization of caveolae. J. Cell Biol. 1994; 127:1199-215.
38. Isshiki M, Ando J, Korenaga R, Kogo H, Fujimoto T, Fujita T, Kamiya A. Endothelial Ca2+ waves preferentially originate at specific loci in caveolin-rich cell edges. Proc Natl Acad Sci USA. 1998; 95:5009-14.
39. Mundy D I, Machleidt T, Ying Y S, Anderson R G, Bloom G S. Dual control of caveolar membrane traffic by microtubules and the actin cytoskeleton. J Cell Sci. 2002; 115:4327-39.
40. Uehara K, Miyoshi M. Tubular invaginations with caveolae and coated pits in the sinus endothelial cells of the rat spleen. Histochem Cell Biol. 1999; 112:351-8.
41. Takiguchi M, Mori M. In vitro analysis of the rat liver-type arginase promoter. J Biol. Chem. 1991; 266:9186-93.
42. Davignon J, Ganz P. Role of endothelial dysfunction in atherosclerosis. Circulation. 2004; 109:E127-32.
43. Inoue K, Arai Y, Kurihara H, Kita T, Sawamura T. Overexpression of lectin-like oxidized low-density lipoprotein receptor-1 induces intramyocardial vasculopathy in apolipoprotein E-null mice. Circ Res. 2005; 97: 176-84.

### INCORPORATION BY REFERENCE

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of treating or preventing atherosclerotic disease in a subject comprising:
administering to the subject an effective amount of a compound inhibits the expression of Arginase II, the activity of Arginase II, or level of free of Arginase II;
thereby treating or preventing atherosclerotic disease in a subject.

2. The method of claim 1, wherein the compound inhibits the level of free Arginase II.

3. The method of claim 2, wherein the compound inhibits the level of free Arginase II by inhibiting the dissociation of Arginase II from microtubules.

4. The method of claim 3, wherein the compound is a microtubule stabilizing agent.

5. The method of claim 4, wherein the compound is selected from the group consisting of paclitaxel, Doublecortin, epothilone, Laulimalide, Vincristine and Epothilone B.

6. The method of claim 3, wherein the compound is an antibody.

7. The method of claim 3, wherein the level of free Arginase II is inhibited by decreasing the amount of oxLDL in a cell.

8. The method of claim 1, wherein the compound decreases the transcription or translation of Arginase II.

9. The method of claim 8, wherein the compound decreases the translation of Arginase II.

10. The method of claim 9, wherein the compound is a nucleic acid molecule.

11. The method of claim 10, wherein the nucleic acid molecule is an antisense RNA molecule, a siRNA molecule or a shRNA molecule.

12. The method of claim 11, wherein the nucleic acid molecule is an siRNA molecule comprising the sequence set forth as SEQ ID NO:3.

13. The method of claim 1, wherein the compound inhibits the activity of Arginase II.

14. The method of claim 13, wherein the compound is a small molecule, peptide, polypeptide, or nucleic acid molecule.

15. The method of claim 1, wherein the atherosclerotic disease is oxLDL dependent atherosclerotic disease.

16. A method of treating a subject having endothelial dysfunction comprising;
administering to the subject an effective amount of a compound inhibits the expression of Arginase II, the activity of Arginase II, or level of free of Arginase II;
thereby treating a subject having endothelial dysfunction.

17.-30. (canceled)

31. The method of claim 15, wherein the inhibition of Arginase II results in a decrease in nitric oxide (NO) production.

32. A method of determining if a subject is at risk of developing atherosclerotic disease comprising:
obtaining a biological sample from the subject;
determining the level of free Arginase II in the sample;
wherein an elevated level of free Arginase II in the sample as compared to a control is indicative that the subject is at risk of developing atherosclerotic disease.

33.-46. (canceled)

47. A kit for the diagnosis of atherosclerotic disease comprising an antibody specific for Arginase II, and instructions for use.

48. The kit of claim 47, wherein the antibody further comprises a detectable label.

49. The kit of claim 47, further comprising an antibody for tubulin.

50. The kit of claim 49, wherein the antibody further comprises a detectable label.


SEQUENCE LISTING (6 sequences)

SEQ ID NO: 1 - DNA, 19 bp, Artificial Sequence
  Description of Artificial Sequence Synthetic primer
ggcaaggtggcagaagtca

SEQ ID NO: 2 - DNA, 20 bp, Artificial Sequence
  Description of Artificial Sequence Synthetic primer
tggttgtcagtggagtgttg

SEQ ID NO: 3 - DNA, 22 bp, Artificial Sequence
  Description of Artificial Sequence Synthetic primer
ctatcagcactggatcttgttg

SEQ ID NO: 4 - DNA, 22 bp, Artificial Sequence
  Description of Artificial Sequence Synthetic primer
gggagtaggaagttggtcatag

SEQ ID NO: 5 - DNA, 20 bp, Artificial Sequence
  Description of Artificial Sequence Synthetic primer
cggcgacgacccattcgaac

SEQ ID NO: 6 - DNA, 23 bp, Artificial Sequence
  Description of Artificial Sequence Synthetic primer
gaatcgaaccctgattccccgtc